(19) United States
(12) Patent Application Publication
(10) Pub. No.: US 2023/0390510 A1
(43) Pub. Date: Dec. 7, 2023

(54) SYSTEMS AND METHODS FOR ASSESSING CEREBROVASCULAR REACTIVITY

(71) Applicants: THETRUSTEES OF THE UNIVERSITY OF PENNSYLVANIA, Philadelphia, PA (US); DREXEL UNIVERSITY, Philadelphia, PA (US)

(72) Inventors: Ramon DIAZ-ARRASTIA, Philadelphia, PA (US); Kurtulus IZZETOGLU, Bryn Mawr, PA (US); Pratusha REDDY, Philadelphia, PA (US); My Duyen LE, Philadelphia, PA (US)

(73) Assignees: THETRUSTEES OF THE UNIVERSITY OF PENNSYLVANIA, Philadelphia, PA (US); DREXEL UNIVERSITY, Philadelphia, PA (US)

(21) Appl. No.: 18/452,502

(22) Filed: Aug. 18, 2023

Related U.S. Application Data
(63) Continuation of application No. PCT/US2022/017214, filed on Feb. 22, 2022.
(60) Provisional application No. 63/152,109, filed on Feb. 22, 2021.

(51) Int. Cl. A61M 16/00; A61M 16/20; A61M 16/06; A61M 16/08; A61M 16/12

(52) U.S. Cl. CPC A61M 16/0045; A61M 16/202; A61M 16/06; A61M 16/0875; A61M 16/125; A61M 2230/432

(57) ABSTRACT

The disclosed subject matter provides a device and a method for inducing and sustaining hypercapnia of a subject. The device can include a tube, a face mask for breathing of the subject, a gas collection reservoir for collecting an expired gas from the subject, a valve chamber, a sensor for detecting a carbon dioxide level from the expired gas from the subject, and a controller configured to control a partition of the valve chamber for changing a gas composition in the valve chamber based on the readings of the sensor. The valve chamber can include a first opening, a second opening, and a third opening, wherein the first opening can be configured to allow an external air flowing into the valve chamber, and the expired gas flowing out from the valve chamber, wherein the second opening, coupled to the face mask through the tube, can be configured to allow a gas passage between the subject and the valve chamber, wherein the third opening, coupled to the gas collection reservoir though the tube, can be configured to allow a gas passage between the valve chamber and the gas collection reservoir.

### CROSS-REFERENCE TO RELATED APPLICATION

This application is a Continuation of International Patent Application No. PCT/US2022/017214, filed on Feb. 22, 2022, which claims priority to U.S. Provisional Patent Application No. 63/152,109, which was filed on Feb. 22, 2021, the entire contents of which are incorporated by reference herein.

### BACKGROUND

The brain is dependent on the precise coupling of cerebral blood flow (CBF) to metabolic demand as a consequence of its metabolic rate and limited capacity for substrate storage. Cerebrovascular reactivity (CVR), the ability of cerebral vessels to dilate or constrict in response to challenges, can be an important index of the health of the brain's microvasculature and provide information regarding the available vascular reserve that is complementary to measuring steady-state parameters such as CBF. Microvascular insufficiency, as measured by deficits in CVR, can be a prominent feature in a wide range of neurologic disorders, is prognostic of an unfavorable natural history, and identifies patients who may benefit from targeted therapies. Impaired CVR has been implicated in ischemic stroke, multiple sclerosis, migraine headache, leukoaraiosis, Parkinson's disease, Alzheimer's disease, and age-related cognitive impairment.

Microvasculopathy can be an attractive target for therapeutic interventions aimed at improving neurologic function or slowing neurodegeneration, as established pharmacologic and non-pharmacologic therapies with proven efficacy in improving vascular health are readily available. Such therapies can include PDE-5 inhibitors, HMG-CoA reductase inhibitors, angiotensin II receptor blockers, erythropoietin transforming growth factor-beta (TGF) inhibitors, aerobic exercise, dietary interventions, and nutraceuticals. The availability of these therapies highlights the need for reliable methods to assess CVR in a broad range of clinical settings, including the Emergency Department (ED), the neurological intensive care unit (NICU), and outpatient clinics. For example, this clinical need is recognized by the American Medical Association (AMA) through the establishment of Current Procedural Terminology (CPT) Code 93890 for “Study of Vasomotor Reactivity with Transcranial Doppler.” Despite this clinical need, certain techniques to measure CVR are not sufficiently used in routine clinical practice, primarily due to the lack of safe, reliable, inexpensive, and convenient methods to administer vasoactive stimuli. Although certain techniques (e.g., inducing hypercapnia by breath-holding or administering a fixed inspired fractional concentration of CO₂) can be used for measuring CVR, these techniques can require bulky and expensive types of equipment and fail to provide a consistent change in end-tidal CO₂ (EtCO₂), and measurement of the partial pressure of arterial carbon dioxide (paCO₂).

Therefore, there is a need for improved devices and techniques for assessing CVR.

### SUMMARY

The disclosed subject matter provides techniques for inducing and sustaining hypercapnia of a subject. An example device can include a tube, a face mask for mouth breathing of the subject, a gas collection reservoir for collecting an expired gas from the subject, a valve chamber, a sensor for detecting a carbon dioxide level from the expired gas from the subject, and a controller configured to control a partition of the valve chamber for changing a gas composition in the valve chamber based on the readings of the sensor. The valve chamber can include a first opening, a second opening, and a third opening. The first opening can be configured to allow external air flowing into the valve chamber, and the expired gas flowing out from the valve chamber. The second opening, coupled to the face mask through the tube, can be configured to allow a gas passage between the subject and the valve chamber. The third opening, coupled to the gas collection reservoir through the tube, can be configured to allow a gas passage between the valve chamber and the gas collection reservoir.

In certain embodiments, the tube can be a corrugated plastic tubing. In non-limiting embodiments, the face mask can be configured for the nose or mouth breathing of the subject. In some embodiments, the sensor can be coupled to a capnometer. In non-limiting embodiments, the valve chamber can be coupled to a motor system.

In certain embodiments, the gas collection reservoir can have a capacity of about 5 liters. In non-limiting embodiments, the gas collection reservoir can include a manual valve that can be configured to control gas flow to and/or from the gas collection reservoir.

In certain embodiments, the controller can be an adaptive proportional-integral-derivative (PID) controller. In non-limiting embodiments, the controller can provide a closed-loop feedback based on the reading of the sensor. In some embodiments, the controller can be configured to control the partition of the valve chamber through the motor system.

In certain embodiments, the disclosed device can induce the hypercapnia of the subject by increasing a partial pressure of arterial carbon dioxide (paCO₂) of the subject by about 10 mmHg. In non-limiting embodiments, the device can sustain the hypercapnia for about 25 seconds.

The disclosed subject matter provides a device for inducing and sustaining hypercapnia of a subject using manual valves. An example device can include a face mask for nose or mouth breathing of the subject, a carbon dioxide reservoir, and a tube, a first manual valve that connects the carbon dioxide reservoir and the tube, and a second manual valve that connects the tube and the external environment. The tube can connect the face mask, the carbon dioxide reservoir, and an external environment for allowing an inspiration of gas from the external environment, the carbon dioxide reservoir, or a combination thereof.

In certain embodiments, the first manual valve and the second manual valve are manual ball valves. In non-limiting embodiments, the first manual valve can be configured to be opened, and the second manual valve can be configured to be closed for inducing hypercapnia of the subject. In some embodiments, the second manual valve can be configured to be partially opened to dilute carbon dioxide-rich air inspired by the subject for maintaining the hypercapnia of the subject.

In certain embodiments, the tube can be a dual-purpose tube.

In certain embodiments, the disclosed device can induce the hypercapnia of the subject by increasing a partial pressure of arterial carbon dioxide (paCO₂) of the subject by about 10 mmHg. In non-limiting embodiments, the device can sustain the hypercapnia for about 25 seconds.

The disclosed subject matter provides methods for inducing and sustaining hypercapnia of a subject. An example method can include filing a gas collection reservoir at a predetermined gas concentration by allowing the subject to breathe in room air and to expire air to the gas collection reservoir through a face mask, instructing the subject to rebreathe the expired air from the gas collection to induce hypercapnia of the subject, and measuring a concentration of carbon dioxide in inspired air of the subject.

The method can further include mixing the air room air and expired air using a valve chamber to achieve the predetermined gas concentration.

In certain embodiments, the method can further include mixing the air room air and expired air using a tube with a manual valve to achieve the predetermined gas concentration.

In certain embodiments, the method can further include assessing cerebrovascular reactivity of the subject during the hypercapnia using an imaging analysis device. In non-limiting embodiments, the cerebrovascular reactivity can be assessed by detecting changes of cerebral blood flow (CBF) of the subject.

In certain embodiments, the hypercapnia can be induced by increasing a partial pressure of arterial carbon dioxide (paCO₂) of the subject by about 10 mmHg. In non-limiting embodiments, the hypercapnia can be sustained for about 25 seconds.

The disclosed subject matter will be further described below.

### BRIEF DESCRIPTION OF THE DRAWINGS

FIGS. 1A-1B provide a diagram and a photograph of an example system for assessing cerebrovascular reactivity in accordance with the disclosed subject matter.

FIG. 2 provides images of an example respiratory valve in accordance with the disclosed subject matter.

FIGS. 3A-3C provide schematic diagrams showing the operation of the closed-loop hypercapnia administration device in accordance with the disclosed subject matter.

FIG. 4 provides a graph showing an example respiratory protocol for the disclosed system in accordance with the disclosed subject matter.

FIG. 5 provides schematic diagrams showing an example device with manual ball valves in accordance with the disclosed subject matter.

FIG. 6 provides a graph showing the end-tidal CO₂ (EtCO₂) changes during hypercapnic and normocapnic states induced by a Douglas bad method, a rebreathing device using manual control, and an automated proportional controller system in accordance with the disclosed subject matter.

FIG. 7 provides a graph showing hypercapnia to about 10 mmHg and normocapnia to 0 mmHg induced by the disclosed device with manual ball valves in accordance with the disclosed subject matter.

FIG. 8 provides a graph showing the sustentation of hypercapnia induced by the disclosed device with manual ball valves in accordance with the disclosed subject matter.

FIG. 9 provides graphs showing the within-session (across rows) and between section variability (across columns) of hypercapnia induced by the disclosed device with manual ball valves in accordance with the disclosed subject matter.

FIG. 10 provides a diagram showing an example closed-loop controller system in accordance with the disclosed subject matter.

It is to be understood that both the foregoing general description and the following detailed description are exemplary and are intended to provide further explanation of the disclosed subject matter.

### DETAILED DESCRIPTION

The disclosed subject matter provides techniques for assessing the administering hypercapnia, a potent and convenient vasodilatory stimulus which is used to measure CVR. The disclosed techniques can induce hypercapnia in a subject without bulky and expensive equipment (e.g., gas mixture) by utilizing the subject's exhaled (hypercapnic) air and room air (atmospheric air). With the size and portability of the device, the disclosed subject matter can allow bedside evaluations of CVR in patients with a wide range of neurologic disorders (e.g., traumatic brain injury, cerebral atherosclerosis, aging, and dementia).

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

As used herein, the word “coupled” means directly connected together or connected through one or more intervening elements. The connection can be a physical connection or an operable connection (e.g., a signal or wave can be transmitted between the elements).

An “individual” or “subject” herein is a vertebrate, such as a human or non-human animal, for example, a mammal. Mammals include, but are not limited to, humans, primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

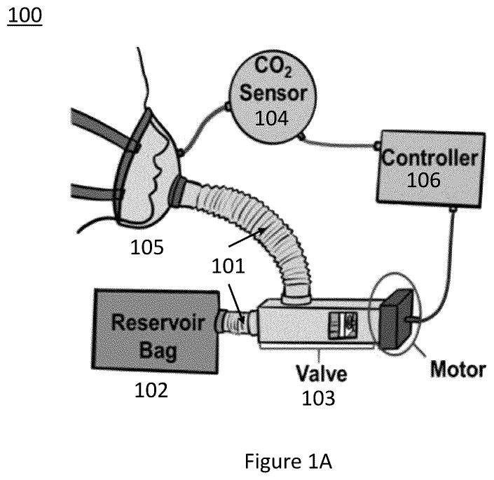

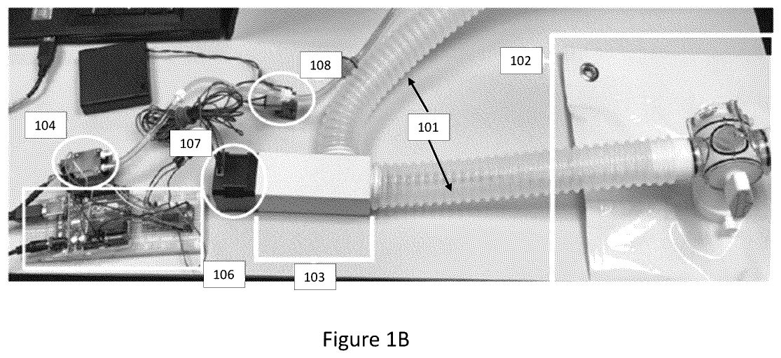

In certain embodiments, the disclosed subject matter provides a device for administering hypercapnia. As shown in FIGS. 1A and 1B, an example device 100 can include a tube 101, a gas collection reservoir 102, a valve chamber 103, a sensor 104, a face mask 105, a controller 106, a motor 107, and/or an air pump 108.

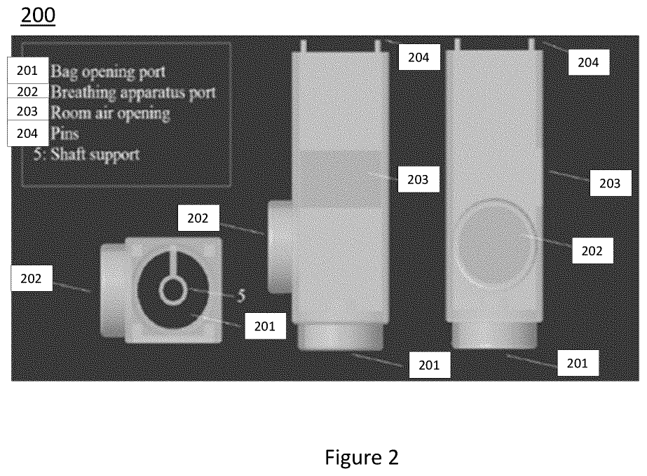

In certain embodiments, as shown in FIG. 2, the valve chamber 103 can be coupled to the motor. In non-limiting embodiments, the valve chamber can be a respiratory valve (e.g., 3-way respiratory valve). The respiratory valve can include at least one opening and a holder for a motor rod and a piston. For example, as shown in FIG. 2, the 3-way respiratory valve chamber 200 can include three openings (e.g., one to the CO₂ reservoir 201, one to the breathing tube 202, and one to room air 203) and a holder (e.g., pin) for a motor/piston 204. The respiratory valve 200 can be a 3-way respiratory valve automated by a controller. The opening 203 can be a one-way passage that acts as the source of fresh air. The opening 202 can be a 2-way passage that either directs inspired air to the breathing mask or directs expired air to the gas collection reservoir. The opening 201 can connect a corrugated flex tube that feeds into the gas collection reservoir to store expired air. The third opening 201 can bring expired air into the valve for rebreathing.

In certain embodiments, the respiratory valve can be an automated electromechanical flow control valve. For example, the device can include at least one closed pneumatic solenoid valves and a dilution port. As the solenoid valve being used works on ‘ON/OFF’ functionality, the fractional opening of the manual valve that can result in dilution of the CO₂ concentrated air can be performed via the addition of a dilution port. The dilution port can be placed in series with the valve controlling airflow from the CO₂ reservoir that can be open during use of the device, ensuring that a controlled amount of outside air can be let in while the device is being used in the hypercapnic position. This dilution port can be the “leak” in the leaky bag. In this position, exhaled air can be both expelled from the dilution port and held in the CO₂ reservoir. Upon inhalation, air can be drawn simultaneously from the CO₂ reservoir and the dilution port so that stored air from the reservoir and fresh outside air can mix.

In certain embodiments, the collection reservoir can be a non-diffusing gas collection bag with a capacity of 1, 2, 3, 4, 5, 6, 7, 8, 9, or 10 liters. It can act as a gas reservoir to collect expired gas from the patient through opening 201 and to deliver gas for rebreathing. In non-limiting embodiments, the collection reservoir can include a manual valve. The manual valve can open or close the reservoir. For example, the manual valve closes the reservoir when the patient is breathing in only fresh air from opening 201.

In certain embodiments, the mask can be a rebreather mask that can be used for nose breathing. The rebreather mask can be connected to the opening 202 that can direct either fresh air or mixed re-breathed air to the mask and direct expired air from the mask back into the respiratory valve.

In certain embodiments, the valve chamber, the face mask, and a gas collection reservoir can be coupled through the tube. In non-limiting embodiments, the tube can be a corrugated plastic clear flex tubing. For example, the corrugated plastic clear flex tubing can be used for the openings to allow for the passage of inspired and expired gas from the subject.

In certain embodiments, the sensor can be a carbon dioxide sensor with a capnometer that can be connected to the mask from where expired air can be pumped to and analyzed in the sensor. In non-limiting embodiments, the sensor can include or be coupled to a capnometer, which can be connected to a computer with a USB connector. The capnograph can be seen in real-time with a capnography program installed in the computer or the controller. The computer or the controller can also be used to control the valve opening and closing via the controller circuit by moving the partition of the valve chamber.

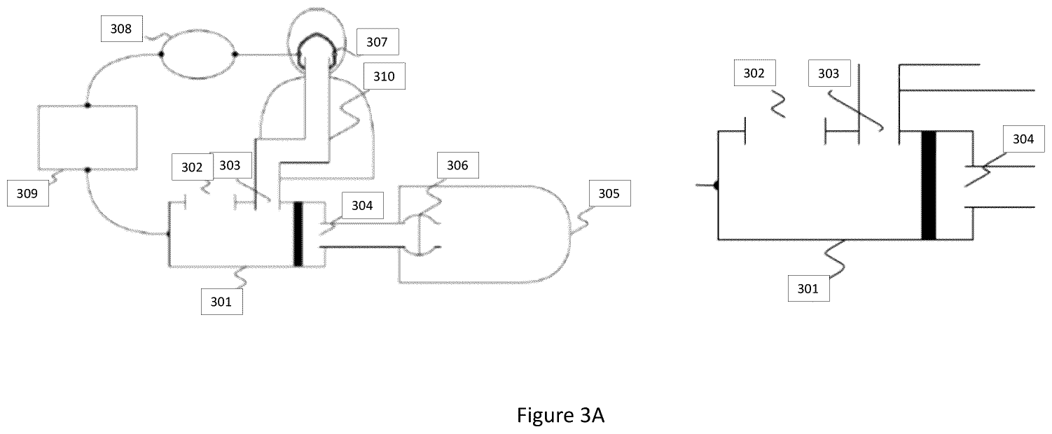

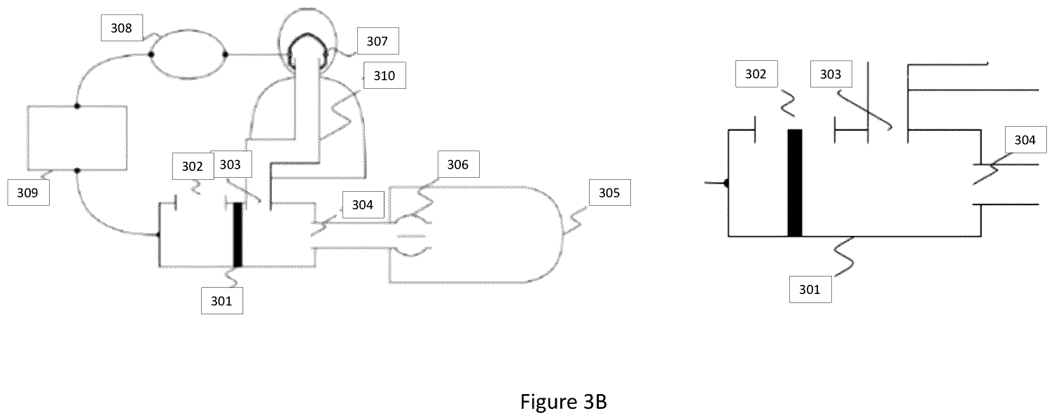

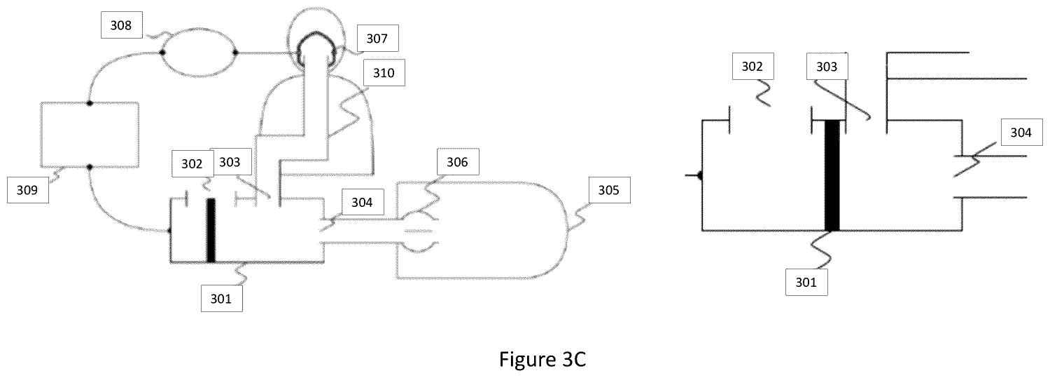

In certain embodiments, the disclosed device can further include a controller circuit. In non-limiting embodiments, the disclosed device can include an adaptive proportional-integral-derivative (PID) including a controller circuit 309 that can controls the respiratory valve 301 by moving a partition (i.e., solid black partition in images in FIGS. 3A-3C) to control what air composition the patient is breathing/rebreathing through the face mask 307. For example, at the start of induced hypercapnia, the subject can be rebreathing from the gas collection bag 305, allowing a rapid rise in the end-tidal CO₂ (EtCO₂). After the target partial pressure of arterial carbon dioxide (paCO₂) plateau is reached, the piston can be positioned where sufficient room air can be allowed to maintain paCO₂ at the desired plateau until the end of the hypercapnia period. Sufficient room is defined as the volume of room air needed to mainintain 10 mmHg change in EtCO₂ values.

In non-limiting embodiments, the controller circuit can incorporate or be coupled to the CO₂ readings 308 from the sensor in the control system to determine the movement of the partition. For example, the mixing of the room air and expired gas can be achieved by the PID controller. The CO₂ sensor 308 can acquire EtCO₂ readings from the subject and send the readings to a microprocessor, which in turn can change the location of the piston within the chamber for changing the composition of the gases delivered to the subject. In some embodiments, the process of increasing or decreasing the subject's EtCO₂ can be achieved by a PID controller 309, where the difference between the subject's EtCO₂ values and desired increase or decrease can be used to determine the required position change. By moving the piston, the airflow can be directed to come from only the CO₂ reservoir 305 (through opening 304), from only the ambient air in the room (through an opening 302), or a mixture of both.

In certain embodiments, the disclosed device can include a hydrophobic filter to remove moisture from the system.

In certain embodiments, the disclosed subject matter provides methods of using the disclosed devices for administering hypercapnia. The method can include filing a gas collection reservoir at a predetermined gas concentration by allowing the subject to breathe in room air and to expire an air to the gas collection reservoir through a face mass, instructing the subject to rebreathe the expired air from the gas collection, and measuring a concentration of carbon dioxide in an inspired air of the subject.

The method can include allowing a subject to breathe through the mask 307 of the disclosed device. For example, a subject can wear the face mask 307 and be allowed to get comfortable breathing through the mask 307. At this stage, as shown in FIG. 3A, the partition position of the valve chamber allows only room air to flow in from the first opening 302 and expired air to flow out of the first opening 302. In non-limiting embodiments, the manual valve 306 of the gas collection bag can be closed. The subject can inspire room air with an anticipated gas concentration (e.g., about 0.04% CO₂, about 21% O₂, and about 79% N₂).

In certain embodiments, the method can further include filling the gas collection reservoir at a predetermined gas concentration by allowing the subject to breathe in room air and expire air to the gas collection reservoir. For example, the first opening 302 is opened at the position shown in FIG. 3B, and the subject can breathe in room air, and the expired air is directed and filled in the gas reservoir 305 through the third opening 304. This can be repeated until the reservoir is filled. Then, the third opening 304 can be closed, and the subject can continue to breathe in room air and exhale through the first opening 302. The anticipated gas concentration in the reservoir can be more than about 5% CO₂, less than about 16% O₂, and about 79% N₂.

In certain embodiments, the method can further include allowing the subject to rebreathe the expired air from the gas collection reservoir and measuring a concentration of CO₂ in the inspired air. For example, the subject can rebreathe the previously expired air from the gas reservoir 304 at the valve position as shown in FIG. 3C. The first valve opening 302 can be fully closed in this position to stop room air from coming in. The subject only breathes in the previously expired air with anticipated gas concentration (e.g., more than about 5% CO₂, less than about 16% 02, and about 79% N₂) through the face mask 307 connected to valve 301 through a tube 310. The CO₂ sensor can measure the percentage of CO₂ in the inspired air.

In certain embodiments, the method can further include diluting the expired air using the room air in the gas collection reservoir to allow the subject to inspire air at the predetermined gas concentration. For example, the air room air and expired air can be mixed using a valve chamber to achieve the predetermined gas concentration. The controller 309 can adjust the partition position so that room air can come into the valve. The valve can be in at the valve position shown in FIG. 3B, where the room air dilutes the previously expired air coming from the reservoir such that the inspired air composition can be in a predetermined concentration ranges (e.g., about 5% CO₂, about 16% O₂, and about 79% N₂). In non-limiting embodiments, the air room air and expired air can be mixed using a tube with the disclosed manual valve to achieve the predetermined gas concentration.

In certain embodiments, the subject's hypercapnic state can be maintained for at least 60 seconds using the disclosed device. The disclosed device can also return a subject's EtCO₂ to baseline state by diluting the expired air in the collection reservoir with the room air.

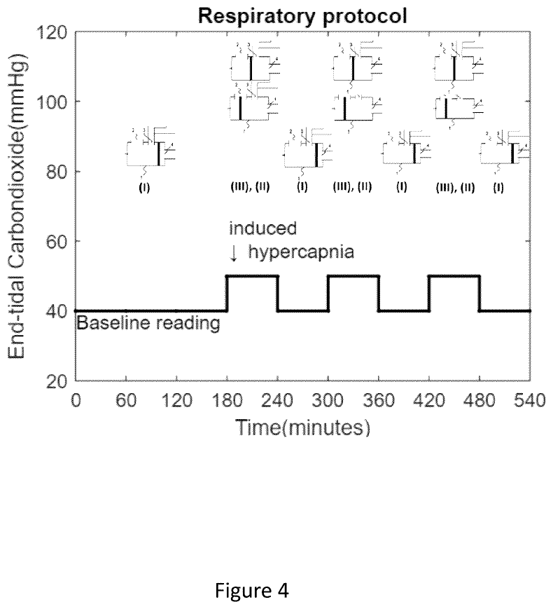

In non-limiting embodiments, the method can be repeated to induce multiple hypercapnic states. For example, as shown in FIG. 4, after this hypercapnic stage, the valve can be switched back to valve position I to allow the end-tidal CO₂ level to drop back to baseline for the next 60 seconds.

In certain embodiments, the method can further include detecting changes of cerebral blood flow (CBF) during the hypercapnia induced by the disclosed subject matter using an imaging analysis device (e.g., magnetic resonance imaging).

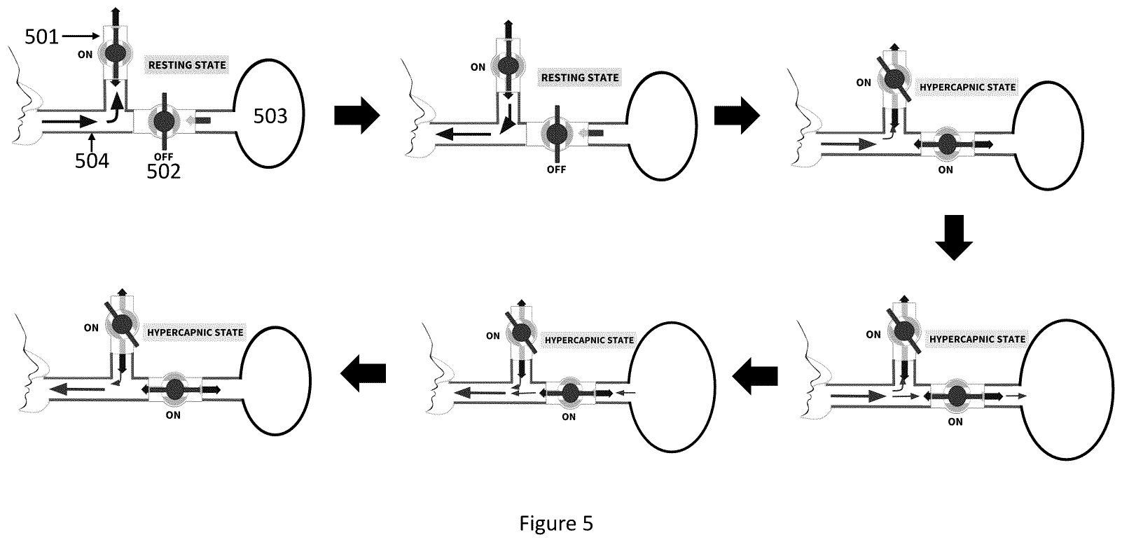

In certain embodiments, the disclosed device can be used without the valve chamber. For example, the valve chamber can be replaced with a manual valve shown. In non-limiting embodiments, the valve can be a manual ball valve. As shown in FIG. 5, the valves 501, 502 can be manually controlled by an operator, who monitors the end-tidal CO₂ (EtCO₂) readings and opens or closes the valves to maintain EtCO₂ at baseline (e.g., approximately 40 mm Hg) or mild hypercapnia (e.g., 10 mm Hg over baseline). In non-limiting embodiments, the disclosed device with the manual valve can mix a patient's own exhaled air to room air to obtain desired levels of CO₂. The disclosed device can be connected to a capnograph for continuous CO₂ monitoring, a collapsible bag 503 capturing exhaled air, a connecting tube 543 equipped with a bag air valve 501 and a room air 502 valve controlling, and a hydrophobic filter to remove moisture from the system. The CO₂ levels can be manipulated by the user with the 2 valves, which control the proportion of exhaled air to room air. By manually controlling the valve, the operator can purposefully and reliably induce mild hypercapnia (e.g., a 10 mm Hg increase in EtCO₂) in a subject with the use of their own exhaled air as the CO₂ source. The outside air can be a dilution factor to control CO₂ concentration levels in the inhaled air of the user. Through the use of flow valves and mixture constants, the device can accurately provide precise levels of CO₂ concentrated air to the user without external air tanks and/or a large reservoir to store this air mixture. In non-limiting embodiments, the disclosed device can assess CVR with human user's exhaled air and room air (atmospheric air). The concentration of inhaled CO₂ can then be manipulated by the user through the leak valve when the device is in use.

In certain embodiments, the disclosed subject matter can be used to diagnose a neurologic disorder (e.g., traumatic brain injury, cerebral atherosclerosis, aging, and dementia). For example, induced hypercapnia, i.e., a increase of partial arterial carbon dioxide pressure (paCO₂), can be used to increase cerebral blood flow (CBF) to assess CVR. In healthy individuals, increasing paCO₂ by about 10 mmHg can increase CBF by 5 to 20%. The disclosed device can be used to induce hypercapnia of the subject by increasing paCO₂ of the subject about 10 mmHg and sustain the hypercapnia for about 25 seconds. Subjects having suffered a traumatic brain injury (TBI), Alzheimer's disease, or multiple sclerosis can show decreased CBF following hypercapnia correlated with poor clinical outcome.

In certain embodiments, an imaging device (such as magnetic resonance imaging (MRI), transcranial Doppler (TCD), or near-infrared spectroscopy (NIRS)) can be further used for assessing CVR.

### EXAMPLES

### Example 1: Practical Method for the Delivery of Controlled Hypercapnia for the Assessment of Cerebrovascular Reactivity

Traumatic brain injury (TBI) is one of the major causes of disability in the United States, affecting 1.5 million people yearly. TBI often results in damage to the blood vessels in the brain, known as traumatic cerebrovascular injury (TCVI), which is difficult to assess. One widely used method involves increasing a patient's CO₂ levels (hypercapnia), increasing cerebral blood flow (CBF). By artificially elevating the CO₂ levels and using brain imaging technology, including but no limited MRI, TCD, or NIRS, it can assess cerebrovascular reactivity (CVR) response and determine any microvascular damage resulting from a TBI. Existing medical devices designed to induce hypercapnia have limitations because of size and cost. For example, the current clinical standard for inducing hypercapnia is the Douglas Bag method. The volume of the Douglas bag is 500 L, which takes up a very large space in a clinical setting and makes it unsuitable in many situations where it would be highly useful. The requirement for gas tanks further complicates its use in routine clinical settings. Furthermore, the setup of this machine can take upward of 30 minutes and is impractical for local/onsite evaluation of TCVI.

The closed-loop hypercapnia administration devices, which utilize room air and the patient's own expired air as gas mixtures, were developed for improved portability and accuracy. The disclosed devices can avoid the need for gas tanks making this device portable, easy-to-use, and inexpensive. The disclosed portable device can automatically induce periodic hypercapnia to a specific level in response to a patient's end-tidal CO₂ (EtCO₂). The device can administer and maintain a 10 mmHg rise to a patient's EtCO₂, which corresponds with the 5% CO₂ stimulus usually used in TBI assessment. The device can also return the patient to their baseline EtCO₂ level. The disclosed device can be a portable and automated device that can induce periodic hypercapnia, allowing measurement CVR in patients with neurological disorders.

Hypercapnia Rebreather Device with Manual Ball Valves

The device can include (i) a dual-purpose tube that can allow the inspiration of gases from room air, CO₂ rich bag air, or both; (ii) a 2 liter CO₂ reservoir; (iii) two manual ball valves that connected to the dual-purpose tube and either the reservoir or environment; and (iv) a face mask that only allowed mouth breathing. A user was able to move the position of the valves (e.g., two ball valves) to elicit breathing of different gas compositions (e.g., room air and CO₂+room air). The opening of the reservoir valve and closure of the room air valve allowed for the buildup of CO₂ concentration and subsequent inducement of hypercapnia. Turning the room air valve to partially open allowed for dilution of the CO₂-rich air being inspired by the subject, thereby resulting in maintenance of the hypercapnic state. The degree to which the valve was opened to room air was dependent upon how much dilution was required per subject. A user was able to identify whether changing the percent opening of the valve connected to the CO₂ or room air is sufficient to increase EtCO₂ changes by about 10 mmHg, to maintain the stimulus at that value for at least 30 seconds, and to re-establish baseline EtCO₂ values using the disclosed device with manual valves.

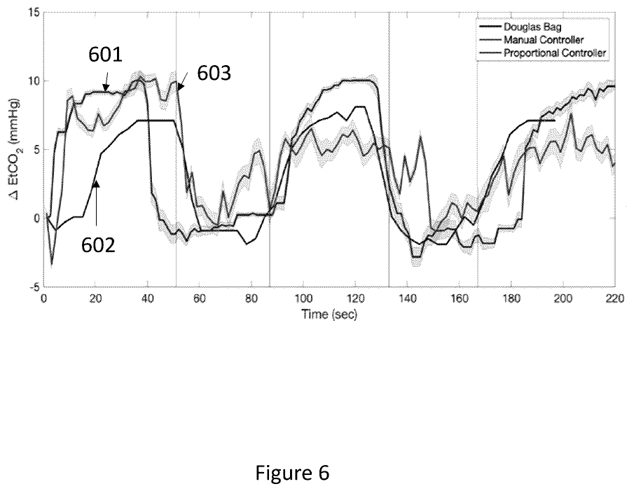

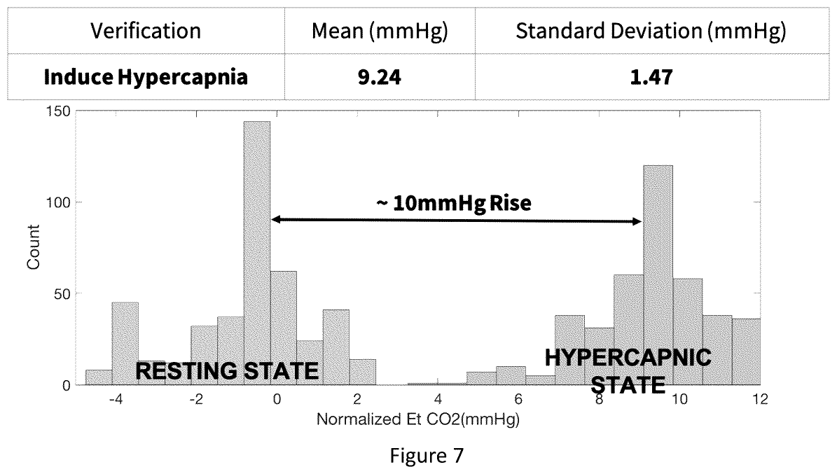

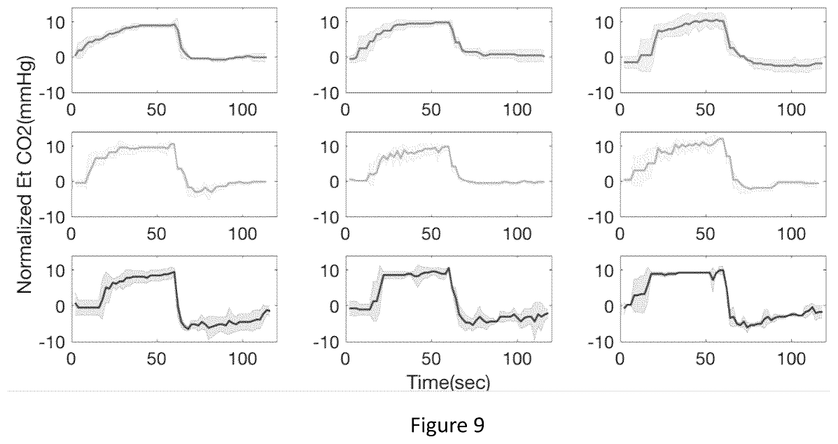

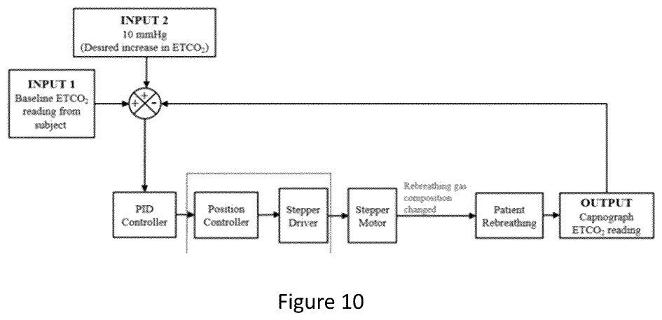

The disclosed device with manual controls was used on three subjects, who underwent three cycles of normocapnia alternating with hypercapnia across three trials. The results (FIG. 6) indicated that (i) an average increase of 9.28+/−1.48 mmHg in EtCO₂ was observed across all hypercapnic segments; (ii) hypercapnic states were maintained within 8 to 12 mmHg for approximately 25 seconds; (iii) EtCO₂ values 601 decreased to −1.73+/−1.85 mmHg during normocapnic phases; (iv) the manual device performed better at targeting 10 mmHg rise in EtCO₂ value than Douglas Bag 602 (FIG. 6). As shown in FIG. 7, an average increase of 9.24 mmHg in EtCO₂ was observed across all hypercapnic segments from the three subjects. The induced hypercapnic states were maintained within 8 to 12 mm Hg for approximately 25 seconds. Comparison of normocapnic to baseline segments displayed that EtCO₂ values were re-establish after hypercapnia, and the coefficient of variation within and across subjects was 10%. As shown in FIG. 9, hypercapnia induced by the disclosed device was repeatable and reproducible across various subjects and sessions

Hypercapnia Rebreather Device with an Automated Respiratory Valve

The device can include: (i) a respiratory valve, which controls the fraction of inspired air coming from either the room or from the rebreather bag; (ii) a gas collection reservoir (e.g., 5-liter capacity); (iii) a rebreather mask; (iv) CO₂ and O₂ sensors; (v) an adaptive proportional-integral-derivative (PID) controller; (vi) a stepper motor system. As the patient breathed, the CO₂ sensor was configured to take EtCO₂ readings from the patient, feed that data to the PID controller, which in turn controlled the flow of air in the system through the movement of the partition via a position controller, which can be embedded in the stepper motor driver. This control was able to allow for the system to adjust to induce hypercapnia, maintain hypercapnia, or return to baseline by directing airflow.

The respiratory valve can include three openings (e.g., one to room air, one to the CO₂ reservoir, and one to the breathing tube) and a holder for a stepper motor rod and a piston. The mixing of the room air and expired gas can be achieved by the PID controller. The CO₂ sensor can acquire EtCO₂ readings from a subject and provide the information to a microprocessor, which in turn will change the location of the piston within the chamber, ultimately changing the composition of the gases delivered to the subject. The process of increasing or decreasing the subject's EtCO₂ can be achieved by a PID controller design, where the difference between the subject's EtCO₂ values and desired increase or decrease can be used to determine the required position change. By moving the piston, the airflow could be directed to come from only the CO₂ reservoir, from only the ambient air in the room, or a mixture of both.

For the disclosed hypercapnia rebreather device with the automated respiratory valve, the manual valves of the hypercapnia rebreather device with manual ball valves were replaced with automated components (e.g., a CO₂ sensor, a microcontroller, a stepper motor system, and a custom-designed respiratory valve). The respiratory valve was designed and developed using a 3D printer.

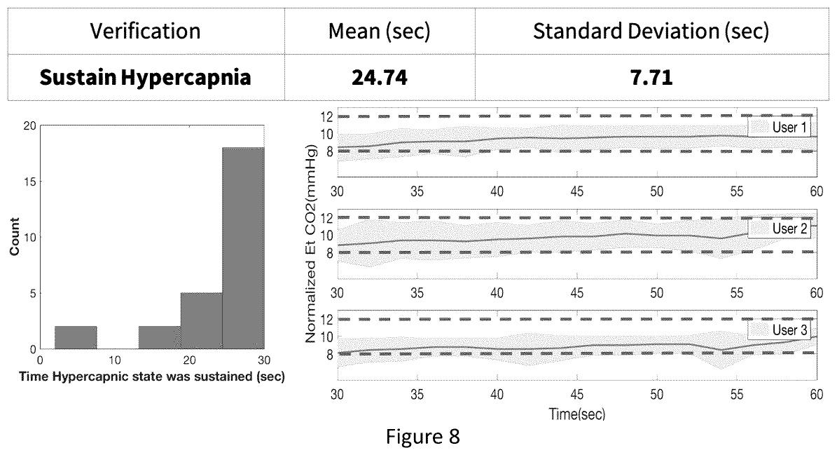

The process of increasing or decreasing the patient's EtCO₂ was done via a closed-loop control system (FIG. 8). The patient had their baseline EtCO₂ (output) level read by a capnometer (sensor). The desired level of EtCO₂ was based on user input; for example, an increase of EtCO₂ of 10 mmHg from their baseline reading. The error was the difference between the patient's baseline readings plus the desired level and the patient's current EtCO₂ reading. This error value was used to determine the movements of the stepper motor piston that determined how much room air was able to flow into the system, which in turn affected the concentration of CO₂ in the air that was being administered to the patient. This model was the basis of the relationship between each part of the system.

The device was designed to administer hypercapnia without inhibiting the normal respiratory function of the subject. The velocity multiplied by the area of the valve openings was equal to or greater than the volume of an exhale. To accommodate normal velocity of airflow during exhalation and inhalation (e.g., 130 m/s), the valve was constructed with an opening area of about 1225 mm². The device had a 5 L reservoir to ensure that it can deliver and hold the 500 mL of air expelled or inhaled during breathing to maintain a normal breathing rate. The device was configured to increase a user's EtCO₂ by 10±2 mmHg from their baseline, return to baseline, and accommodate subject variation (e.g., device's ability to reach different target levels of CO₂ increases, up to about a 15% CO₂ stimulus). The CO₂ sensor used to measure EtCO₂ has a measurement range of 0-20% (e.g., 0 to 152 mmHg of CO₂). Considering the average range of EtCO₂ is about 35-45 mmHg and the target is a 10-mmHg rise, this measurement range of the sensor can capture the expected outputs in addition to accommodating a large range of values for variation. The device was configured to maintain hypercapnia for about 30 seconds within ±2 second range. The CO₂ sensor had a 500 ms sampling rate. This fell under the standard deviation requirement, ensuring a time to EtCO₂ reading sensitive enough for the design. The computer hardware had to be able to connect to both a motor and a sensor. The Arduino Uno (i.e., controller) had to have connections for up to 2 devices. The Arduino Uno has 14 input and output pins, which met the requirement for the solution.

The developed device was an automated valve system connected to an airbag and a breathing apparatus. This is displayed in FIG. 1B. The mechanical mechanism was dependent on the shape of the valve chamber and a piston that moved along the valve chamber. The valve piston changed its position based on the rotations of the motor. The motor was connected to a threaded shaft. This threaded shaft ran along through the valve chamber. The pins in FIG. 2 are attached to corresponding holes on the motor. The pins kept the motor and chamber connected. The shaft protruded from the motor all the way through the shaft support seen in FIG. 2. The piston that moved across the chamber had threads that complement the threads of the shaft. As the shaft rotated, the piston was displaced along the shaft. The shape of the chamber prevented the piston from rotating. Depending on the direction of the rotation, the piston either moved towards or away from the motor. The valve was composed of a 3D-printed chamber and a 3D-printed piston. FIG. 2 shows the semitransparent 3D model from 3 different directions. The complete system was composed of a motor, the valve chamber, the CO₂ sensor with cap adapter, the Arduino microcontroller, reservoir bag, and face mask, which relate to breathing tubes. The CO₂ sensor monitored the EtCO₂ of the patient and fed that data to the Arduino, which in turn controlled the motor to direct airflow. This airflow can either come from the gas in the reservoir bag or from the ambient air in the room through the face mask or both.

The CO₂ measurements of the sensor were compared to the measurement of the capnometer under the same conditions. For the verification of the ability of the device to induce hypercapnia, sustain hypercapnia, and return to baseline, it was tested based on a protocol similar to that for the control (Douglas bag). The device's results were compared to that of the Douglas bag to ensure that the device has comparable functionality. In this test, the patient was exposed to two different cycles: baseline (open to atmosphere) and hypercapnia (closed to atmosphere). Each cycle lasted for 1 minute for a total of 3 cycles each. The readings were measured to determine how the EtCO₂ changed throughout the test. The results from the testing of the Douglas Bag and the device were compared when the device met the target.

The dimension of the device was found to be less than 18 cm×38 cm×27 cm and weighed at 1.5 kg.

The disclosed device with the respiratory valve (e.g., an adaptive proportional controller design) was used on two subjects, who underwent three repetitions each. The results (FIG. 6, 603) provided that adaptive controls can allow for control of change in EtCO₂. Specifically, EtCO₂ increased to about 8.57+/−2.95 mmHg during hypercapnia and fell to −2.05+/−2.22 mmHg during normocapnia regardless of subject and trial. The EtCO₂ values varied between about 7.33 to about 11.36 mmHg for hypercapnia and about 0.00 to about −3.89 mmHg for normocapnia and declined with repetitions (e.g., during hypercapnia repetition one induced 9.25 mmHg change while repetition three induced 6.98 mmHg change).

Verification testing was done to examine if the valve was capable of automatically directing airflow and inducing periodic hypercapnia. The device was programmed to automatically open and close the valve based on the time input from the user and dilute the concentration of CO₂ depending on the reading from the capnometer. The results showed that the device can direct airflow and induce hypercapnia. The results further demonstrate that the valve can be controlled by the Arduino Uno and can be set to work automatically. The valve was able to seal the chambers and sufficiently prevent leakage. This was demonstrated by the elevated levels of CO₂ that were observed during the inducing and maintaining hypercapnia stages of the testing.

Based on the verification results, the device was able to induce periodic hypercapnia automatically. The device was also able to bring the levels to baseline. Due to its size, the device can be used in remote locations where the Douglas bag method is not practival. The disclosed device can provide a portable and compact way to induce periodic hypercapnia in patients with traumatic brain injury. The disclosed device can also provide an easy way to measure CVR in patients with neurological disorders, when coupled with instruments to measure relative changes in cerebral blood flow (CBF), such as MRI, TCD, or NIRS. The small size of the device and its portable design make it extremely useful in emergency medicine, particularly emergency rooms where space is restricted, and the timeliness of diagnosis can play a huge factor in patient prognosis. This device can also be useful for military applications (e.g., head trauma). In addition to inducing hypercapnia to evaluate TCVI, this device can have application in clinical research helping clinicians evaluate vasoactive drug therapies for the treatment of TCVI. For example, a test subject can be given a vasoactive drug, then, with the use of the device coupled with an imaging technique such as fMRI or fNIRS regionalized blood flow can be measured to see if these drugs work as intended a major clinical advancement. To remove phase delays and improve system reliability and precision, the controller design can be modified based on subject-varying parameters (e.g., respiratory rate, tidal volume, and EtO2 need to be added to the controller design.

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed is:

1. A device for inducing and sustaining hypercapnia of a subject comprising:
a tube;
a face mask for breathing of the subject;
a gas collection reservoir for collecting an expired gas from the subject;
a valve chamber including a first opening, a second opening, and a third opening, wherein the first opening is configured to allow an external air flowing into the valve chamber and the expired gas flowing out from the valve chamber, wherein the second opening, coupled to the face mask through the tube, is configured to allow a gas passage between the subject and the valve chamber, wherein the third opening, coupled to the gas collection reservoir through the tube, is configured to allow a gas passage between the valve chamber and the gas collection reservoir;
a sensor for detecting a carbon dioxide level from the expired gas from the subject; and
a controller configured to control a partition of the valve chamber for changing a gas composition in the valve chamber based on the readings of the sensor.

2. The device of claim 1, wherein the tube is a corrugated plastic tubing.

3. The device of claim 1, wherein the face mask is configured for mouth breathing of the subject.

4. The device of claim 1, wherein the gas collection reservoir has a capacity of about 5 liters.

5. The device of claim 1, wherein the gas collection reservoir comprises a manual valve that is configured to control gas flow to and/or from the gas collection reservoir.

6. The device of claim 1, wherein the sensor is coupled to a capnometer.

7. The device of claim 1, wherein the controller is an adaptive proportional-integral-derivative (PID) controller.

8. The device of claim 1, wherein the controller provides a closed-loop feedback based on the reading of the sensor.

9. The device of claim 1, wherein the valve chamber is coupled to a stepper motor system.

10. The device of claim 9, wherein the controller is configured to control the partition of the valve chamber through the stepper motor system.

11. The device of claim 1, wherein the device is configured to induce the hypercapnia by increasing a partial pressure of arterial carbon dioxide (paCO₂) of the subject by about mmHg.

12. The device of claim 1, wherein the device is configured to sustain the hypercapnia for about 25 seconds.

13. A device for inducing and sustaining hypercapnia of a subject comprising:
a face mask for breathing of the subject;
a carbon dioxide reservoir; and
a tube that connects the face mask, the carbon dioxide reservoir, and an external environment for allowing an inspiration of air from the external environment, the carbon dioxide reservoir, or a combination thereof;
a first manual valve that connects the carbon dioxide reservoir and the tube;
a second manual valve that connects the tube and the external environment.

14. The device of claim 13, wherein the first manual valve and the second manual valve are manual ball valves.

15. The device of claim 13, wherein the tube is a dual-purpose tube.

16. The device of claim 13, wherein the first manual valve is configured to be opened and the second manual valve is configured to be closed for inducing hypercapnia of the subject.

17. The device of claim 13, wherein the second manual valve is configured to be partially opened to dilute a carbon dioxide-rich air inspired by the subject for maintaining the hypercapnia of the subject.

18. The device of claim 13, wherein the device is configured to induce the hypercapnia by increasing a partial pressure of arterial carbon dioxide (paCO₂) of the subject by about mmHg.

19. The device of claim 13, wherein the device is configured to sustain the hypercapnia for about 25 seconds.

20. A method for inducing and sustaining hypercapnia of a subject comprising:
filing a gas collection reservoir at a predetermined gas concentration by allowing the subject to breathe in room air and to expire an air to the gas collection reservoir through a face mask;
instructing the subject to rebreathe the expired air from the gas collection to induce hypercapnia of the subject; and
measuring a concentration of carbon dioxide in an inspired air of the subject.

21. The method of claim 20, further comprising mixing the air room air and expired air using a valve chamber to achieve the predetermined gas concentration.

22. The method of claim 20, further comprising mixing the air room air and expired air using a tube with a manual valve to achieve the predetermined gas concentration.

23. The method of claim 20, further comprising assessing cerebrovascular reactivity of the subject during the hypercapnia using an imaging analysis device.

24. The method of claim 23, wherein the cerebrovascular reactivity is assessed by detecting changes of cerebral blood flow (CBF) of the subject.

25. The method of claim 20, the hypercapnia is induced by increasing a partial pressure of arterial carbon dioxide (paCO₂) of the subject by about 10 mmHg.

26. The method of claim 20, the hypercapnia is sustained for about 25 seconds.
